(19) United States
(12) Patent Application Publication
(10) Pub. No.: US 2023/0115452 A1
(43) Pub. Date: Apr. 13, 2023

(54) MANAGING TELEMETRY SESSION WITH IMPLANTABLE DEVICE

(71) Applicant: Medtronic, Inc., Minneapolis, MN (US)

(72) Inventor: Mahipal Reddy, Hyderabad (IN)

(21) Appl. No.: 17/933,760

(22) Filed: Sep. 20, 2022

Related U.S. Application Data
(60) Provisional application No. 63/254,630, filed on Oct. 12, 2021.

(51) Int. Cl. A61B 5/07; A61B 5/00

(52) U.S. Cl. CPC A61B 5/07; A61B 5/002; A61B 2560/0481

(57) ABSTRACT

This disclosure is directed to devices, systems, and techniques for an external device to receive data collected by an IMD implanted in a patient. The IMD may collect physiological data associated with a patient and store the physiological data in memory. The IMD may determine an amount of the physiological data stored in the memory to send to an external device and send, via a wireless connection, the determined amount of the physiological data stored in memory to the external device.

This application claims the benefit of U.S. Provisional Application Ser. No. 63/254,630, filed Oct. 12, 2021, which is entitled “MANAGING TELEMETRY SESSION WITH IMPLANTABLE DEVICE” and is incorporated by reference herein in its entirety.

### TECHNICAL FIELD

The disclosure relates to device communication between two or more devices.

### BACKGROUND

A computing device may be configured to receive communications from an implantable medical device (IMD). IMDs may be surgically implanted in a patient to monitor one or more physiological parameters of the patient and/or deliver therapy to suppress one or more symptoms of the patient. For example, an 1 MB may include a cardiac monitor, be configured to deliver cardiac pacing or another electrical therapy to the patient, and/or be configured to terminate tachyarrhythmia by delivery of high energy shocks. A clinician or patient may use an external device to retrieve information collected by the IMD and/or to configure or adjust one or more parameters of the monitoring and/or therapy provided by the IMD. Typically, the external device connects to the IMD via a wireless connection. In some examples, a wireless connection is established between the external device and the IMD using a Bluetooth Low Energy (BLE) wireless protocol. In such an example, the external device is treated as a central device, and one or more IMDs are treated as peripheral devices.

### SUMMARY

In general, the disclosure is directed to devices, systems, and techniques for an external device to receive data collected by an IMD implanted in a patient. The IMD may periodically send data collected by the IMD to the external device or may send such data in response to the IMD detecting the occurrence of certain conditions in the patient. The external device may also send to the IMD a request for data and may, in response, receive, from the IMD, data collected by the IMD.

The MID may include memory that can store the data collected by the IMD. For example, the memory may include storage space to store the data collected over the last 30 days, the data collected over the last 90 days, and the like. In some examples, when the IMD determines to send data to the external device, such as in response to a request for data from the external device, the IMD may send all of the data collected by the IMD and stored in memory to the external device.

However, sending all of the data stored in memory of the IMD to the external device may cause a large amount of data to be transferred through a low-bandwidth communications link, such as BLE. Transmitting all of the data stored in memory of the ID, which may encompass 30 days or more of data collected by the IMD, may often take up to six minutes or more. In scenarios where there is poor connectivity between the external device and the ID, such poor connectivity may cause re-transmissions, re-requests, and/or connection drops that further increases the amount of time required to successfully transmit all of the data stored in memory of the IMD to the external device.

In accordance with aspects of the present disclosure, an IMD may, in response to determining to send data to an external device, determine whether to send all of the data stored in memory to the external device or whether to send, to the external device, data collected by the IMD since the last successful transmission of data to the external device without sending data that has already been successfully transmitted to the external device. In some examples, the IMD may receive, from an external device, a request for data that indicates whether the request is a request for all of the data stored in the memory of the IMD or whether the request is a request for the data collected by the IMD since the last successful transmission of data to the external device. In some examples, the IMD may determine, based on factors such as the quality of the wireless link between the IMD and the external device, whether to send all of the data stored in the memory of the IMD to the external device or whether to send, to the external device, just the data collected by the IMD since the last successful transmission of data to the external device.

The techniques of this disclosure may provide one or more advantages. For example, by sending, to an external device, just the data collected by the IMD since the last successful transmission of data to the external device, the IMD may decrease the amount of time to transfer data between the IMD and the external device. Decreasing the amount of time to transfer data between the IMD and the external device may increase the chances of the IMD successfully transmitting data to the external device by decreasing the chances of an error occurring in the wireless communications between the IMD and the external device, especially in conditions that may cause wireless connectivity issues. Decreasing the amount of time to transfer data between the IMD and the external device may also decrease the amount of time spent by the IMD and the external device to send and receive data, thereby decreasing the power usage of the IMD and the external device to send and receive data.

In one example, a method includes collecting, by processing circuitry of a medical device, physiological data associated with a patient; storing, by the processing circuitry, the physiological data in memory; determining, by the processing circuitry, an amount of the physiological data stored in the memory to send to an external device; and sending, by the processing circuitry via a wireless connection, the determined amount of the physiological data stored in memory to the external device.

In another example, a medical device configured for wireless communication, wherein the first device comprises: sensing circuitry configured to collect physiological data associated with a patient; memory configured to store the physiological data; communication circuitry configured for wireless communication; and processing circuitry electrically coupled to the communication circuitry, wherein the processing circuitry is configured to: determine an amount of the physiological data stored in the memory to send to an external device; and send, using the communication circuitry and via a wireless connection, the determined amount of the physiological data stored in memory to the external device.

In another example, an apparatus comprises: means for collecting physiological data associated with a patient; means for storing the physiological data; means for determining an amount of the physiological data to send to an external device; and means for sending the determined amount of the physiological data stored in memory to the external device.

In another example, a non-transitory computer-readable storage medium comprising program instructions that, when executed by processing circuitry of a medical device, cause the processing circuitry to: collect physiological data associated with a patient; store the physiological data; determine an amount of the physiological data stored in the memory to send to an external device; and send, via a wireless connection, the determined amount of the physiological data stored in memory to the external device.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient and a heart of the patient, in accordance with one or more techniques of this disclosure.

FIG. 2 is a block diagram illustrating an example configuration of components of an implantable medical device (IMD), in accordance with one or more techniques of this disclosure.

FIG. 3 is a block diagram illustrating an example configuration of components of an example external device, in accordance with one or more techniques of this disclosure.

FIG. 4 is a flow diagram illustrating an example operation in accordance with one or more techniques of this disclosure.

Like reference characters denote like elements throughout the description and figures.

### DETAILED DESCRIPTION

This disclosure describes techniques for an external device to wirelessly receive data collected by an implantable medical device (IMD) implanted in a patient. In some examples, an IMD records one or more physiological signals of a patient, where the one or more physiological signals may be indicative of a medical condition. The IMD may use any combination of electrodes, chemical sensors, temperature sensors, or other sensors to sense the physiological signals and store data indicative of the physiological signals in a memory. In some examples, the IMD delivers therapy, such as cardiac pacing or anti-tachyarrhythmia shocks, and store data indicative of the therapy delivered. In some examples, the IMD stores data regarding the status and performance of the IMD and components thereof, and operational parameters that control the functioning of the IMD, e.g., for sensing and/or delivering therapy.

The IMD may typically collect physiological data associated with a patient over a period of time, such as collect physiological data associated with the patient over the past thirty days. Since the IMD is implanted within the patient, in some cases, the ID may wirelessly communicate with an external device, e.g., to transmit at least some of the physiological data collected by the IMD to an external device for analysis by a clinician. For example, the IMD may wirelessly communicate with an external device via a short-range wireless protocol, such as via Bluetooth Low Energy (BLE), which may support relatively low transfer speeds. As such, it may take a relatively long time, such as six minutes or more, to transfer the entire thirty days of physiological data stored by the IMD to the external device.

Taking such a long time to transfer the physiological data stored by the IMD to the external device may increase the chances of a transmission error, such as a dropped connection or other wireless connection issues, from occurring. Such long transfer times may also drain more of the battery of the IMD and the external device compared with shorter transfer times. Further, because the external device may require that an app designed to communicate with the ID be running in the foreground, the long transfer time may increase the chance that the user of the external device may intentionally or unintentionally interrupt the transfer of physiological data by closing out the app or by shuffling the app to the background of the external device.

In accordance with aspects of the present disclosure, instead of transferring all of the physiological data stored by the IMD to the external device, the IMD and external device may determine to transfer, from the IMD to the external device, just the portion of physiological data stored in the IMD that was collected after the most recent successful transmission of physiological data from the IMD to the external device. For example if the most recent successful transmission of physiological data from the ID to the external device was fifteen days ago, the ID may simply just transfer the physiological data collected by the IMD in the last fifteen days to the external device.

In another example, the IMD may transfer, to the external device, the physiological data collected by the IMD that had never previously been transmitted by the IMD to the external device, along with any new physiological data collected by the IMD since the most recent successful transmission of physiological data from the IMD to the external device. For example, if the most recent successful transmission of physiological data from the IMD to the external device was fifteen days ago, but if the most recent successfully transmitted physiological data did not include physiological data that was collected by the IMD less than sixteen days ago, the IMD may transfer the physiological data collected by the IMD in the last sixteen days to the external device.

By transferring just the portion of physiological data stored in the IMD that was collected after the most recent successful transmission of physiological data and/or the portion of physiological data stored in the IMD that was not previously transmitted from the IMD to the external device, the IMD and the external device may reduce the transfer time of transferring physiological data from the IMD to the external device. Reducing the transfer time of transferring physiological data from the IMD to the external device may reduce the chances of errors that may be introduced during the transfer process and may reduce the battery usage of the IMD and the external device, thereby providing certain technical advantages.

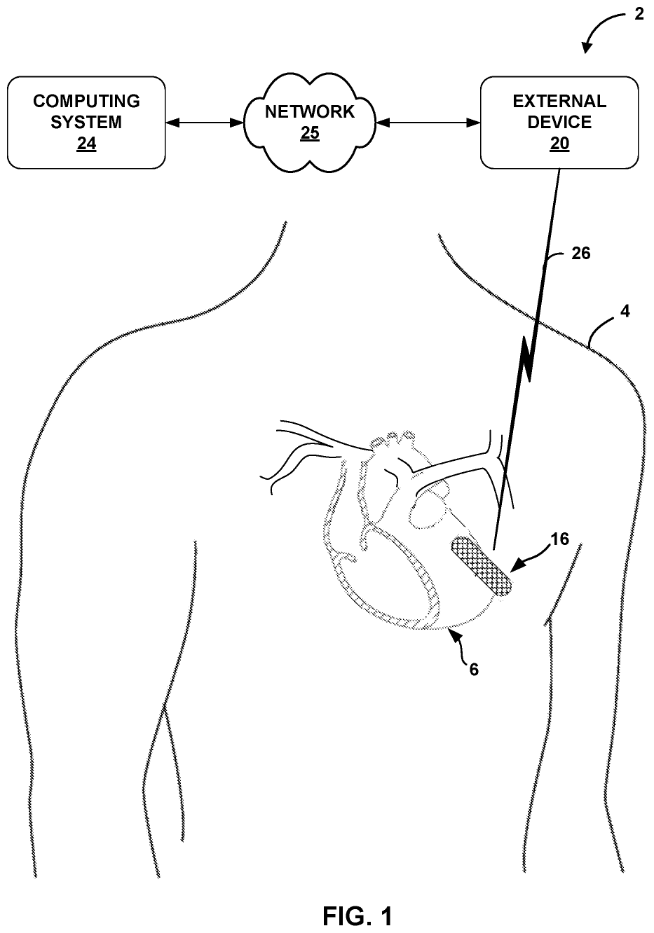

FIG. 1 illustrates the environment of an example medical device system 2 in conjunction with a patient 4 and a heart 6 of patient 4, in accordance with one or more techniques of this disclosure. The example techniques may be used with an IMD 16, which may be in wireless communication with external device 20. External device 20 may also communicate with one or more external computing system(s), such as computing system 24, via network 25.

In some examples, ID 16 is implanted outside of a thoracic cavity of patient 4 (e.g., subcutaneously in the pectoral location illustrated in FIG. 1). IMD 16 may be positioned near the sternum near or just below the level of heart 6, e.g., at least partially within the cardiac silhouette. In some examples, IMD 16 takes the form of a LINQ™ Insertable Cardiac Monitor (ICM), available from Medtronic plc, of Dublin, Ireland.

Clinicians sometimes diagnose patients with cardiac conditions based on one or more observed physiological signals collected by physiological sensors, such as electrocardiogram (ECG) electrodes, electrogram (EGM) electrodes, chemical sensors, or temperature sensors. In some cases, clinicians apply non-invasive sensors to patients in order to sense one or more physiological signals while a patent is in a clinic for a medical appointment. However, in some examples, physiological markers (e.g., irregular heartbeats) of a cardiac condition are rare. As such, in these examples, a clinician may be unable to observe the physiological markers needed to diagnose a patient with a heart condition while monitoring one or more physiological signals of the patient during a medical appointment. In the example illustrated in FIG. 1, IMD 16 is implanted within patient 4 to continuously record one or more physiological signals of patient 4 over an extended period of time and to store such physiological data in the memory of IMD 16.

In some examples, IMD 16 includes a plurality of electrodes. The plurality of electrodes are configured to detect signals that enable processing circuitry of IMD 16 to determine current values of additional parameters associated with the cardiac and/or lung functions of patient 4. In some examples, the plurality of electrodes of IMD 16 are configured to detect a signal indicative of an electric potential of the tissue surrounding the ID 16. Moreover, ID 16 may additionally or alternatively include one or more accelerometers, temperature sensors, chemical sensors, light sensors, pressure sensors, in some examples. Such sensors may detect one or more physiological parameters indicative of a patient condition and may store such physiological data in the memory of IMD 16.

External device 20 is configured to wirelessly communicate with IMD 16 as needed to provide or retrieve information. In some examples, external device 20 acts as an external programming device, e.g., medical device programmer, for IMD 16. External device 20 is an external computing device that a user, e.g., the clinician and/or patient 4, may use to communicate with IMD 16. For example, external device 20 may be a clinician programmer that the clinician uses to communicate with IMD 16 and update one or more settings of IMD 16. Additionally, or alternatively, external device 20 may be a patient programmer that allows patient 4 to control certain operations of IMD 16 and/or view and modify one or more operational parameter values of IMD 16. The clinician programmer may include more programming features than the patient programmer. In other words, more complex or sensitive tasks may only be allowed by the clinician programmer to prevent an untrained patient from making undesired changes to IMD 16.

External device 20 may be a hand-held computing device with a display viewable by the user and an interface for providing input to external device 20 (i.e., a user input mechanism). For example, external device 20 may include a small display screen (e.g., a liquid crystal display (LCD) or a light emitting diode (LED) display) that presents information to the user. In addition, external device 20 may include a touch screen display, keypad, buttons, a peripheral pointing device, voice activation, or another input mechanism that allows the user to navigate through the user interface of external device 20 and provide input. If external device 20 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, external device 20 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device. In some examples, a wireless adapter coupled to the computing device enables external device 20 to establish a wireless communications link 26, such as a Bluetooth Low Energy connection, between the computing device and IMD 16.

When external device 20 is configured for use by the clinician, external device 20 may be used to transmit instructions to IMD 16. Example instructions may include requests to set electrode combinations for sensing and any other information that may be useful for programming into IMD 16. The clinician may also configure and store operational parameters for IMP 16 within IMD 16 with the aid of external device 20. In some examples, external device 20 assists the clinician in the configuration of IMD 16 by providing a system for identifying potentially beneficial operational parameter values.

Whether external device 20 is configured for clinician or patient use, external device 20 is configured to communicate with IMD 16 via wireless communication, such as via communication link 26. External device 20, for example, may communicate via near-field communication technologies (e.g., inductive coupling, NFC or other communication technologies operable at ranges less than 10-20 cm) and far-field communication technologies (e.g., RF telemetry according to the 802.11, Bluetooth, or Bluetooth Low Energy specification sets, or other communication technologies operable at ranges greater than near-field communication technologies).

External device 20 may also be configured to communicate with computing system 24 via network 25. Computing system 24 may comprise computing devices configured to allow a user to interact with IMD 16, or data collected from IMD 16, via network 25. For example, computing system 24 may include one or more handheld computing devices, computer workstations, servers or other networked computing devices. In some examples, computing system 24, network 25, and external device 20 may be implemented by the Medtronic Carelink™ Network or other patient monitoring system.

Network 25 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 25 may include one or more networks administered by service providers, and may thus form part of a large-scale public network infrastructure, e.g., the Internet. Network 25 may provide computing devices, such as external device 20, computing system 24, and IMD 16, access to the Internet, and may provide a communication framework that allows the computing devices to communicate with one another. In some examples, network 25 may be a private network that provides a communication framework that allows computing system 24, IMD 16, and/or external device 20 to communicate with one another but isolates one or more of computing system 24, IMD 16, or external device 20 from devices external to network 25 for security purposes. In some examples, the communications between computing system 24, IMD 16, and external device 20 are encrypted.

In general, IMD 16, and external device 20 may exchange information using at least one communication protocol. Communication protocols define sets of rules that define one or more aspects of data exchange between two or more entities of a network. In some examples, communication protocols are stored as lists of computer-readable instructions and communication protocols may be executed by any combination of hardware (e.g., physical circuitry) and software. An organization, such as a medical device manufacturer, may create its own communication protocols, license communication protocols from a third party, use open source communication protocols, or perform any combination thereof. In some examples, a communication protocol includes security provisions, such as password requirements and data encryption in order to secure the transfer of data between two or more devices in a network.

IMD 16 collects physiological data from patient 4 and stores the collected physiological data associated with patient 4 in memory of IMD 16. The memory of IMD 16 may store a history of the physiological data collected from patient 4. That is, the memory of IMD 16 may store the physiological data collected from patient 4 over a period of time, such as the physiological data collected from patient 4 for the last 30 days, the last 60 days, and the like. As IMD 16 collects additional physiological data from patient 4, IMD 16 may overwrite the oldest physiological data stored in memory in order to maintain, in memory, the physiological data collected from patient 4 over the period of time. For example, if the memory stores the physiological data collected from patient 4 for the last 30 days, IMD 16 may overwrite the physiological data collected earlier than 30 days ago with newly collected physiological data in order to maintain memory as storage for the physiological data collected from patient 4 for the last 30 days.

IMD 16 may send the physiological data associated with patient 4, which may be the physiological data collected by IMD 16 from patient 4 and stored in the memory of IMD 16, over communication link 26 to external device 20. In some examples, IMD 16 may send the physiological data to external device 20 as unencrypted data. In other examples, IMD 16 may encrypt the physiological data and send the encrypted physiological data to external device 20. External device 20 may, in response to receiving the encrypted physiological data, decrypt the encrypted physiological data. IMD 16 may encrypt the physiological data using any suitable encryption technique, such as by using a public encryption key associated with external device 20, a symmetric encryption key associated with external device 20, etc.

In some examples, IMD 16 may periodically send the physiological data collected by IMD 16 from patient 4 and stored in the memory of IMD 16 to external device 20, such as every hour, every day, every two days, every week, and the like. In some examples, IMD 16 may send the physiological data collected by IMD 16 from patient 4 and stored in the memory of IMD 16 to external device 20 in response to detecting the occurrence of certain conditions in patient 4, such as upon the detecting the occurrence of an arrhythmia in the heart of patient 4, upon the blood pressure of patient 4 being above or below a specified threshold, upon the heart rate of patient 4 being above or below a specified threshold, and the like.

In some examples, IMD 16 may send the physiological data collected by IMD 16 from patient 4 and stored in the memory of IMD 16 to external device 20 in response to receiving, from external device 20 and via communication link 26, a request for the physiological data collected by IMD 16. IMD 16 may, in response. In some examples, external device 20 may periodically send requests to IMD 16 for physiological data collected by IMD 16. In some examples, external device 20 may receive user input indicative of a request for physiological data collected by IMD 16 and may, in response to receiving the user input, send a request to IMD 16 for physiological data collected by IMD 16. In some examples, one or more applications executing at external device 20 may call an application programming interface (API) function that causes external device 20 to send a request to IMD 16 for physiological data collected by IMD 16. In some examples, external device 20 may receive, from an external server system such as computing system 24, an indication of a request for physiological data collected by IMD 16 and may, in response to receiving the user input, send a request to IMD 16 for physiological data collected by IMD 16.

IMD 16 may, when sending the physiological data associated with patient 4, determine the amount of physiological data collected by IMD 16 and stored in memory of IMD 16, to send to external device 20, and send the determined amount of physiological data stored in memory to external device 20. In some examples, IMD 16 may determine to send all of the physiological data associated with patient 4 and stored in the memory of IMD 16 to external device 20. For example, if the memory of IMD 16 stores the last 30 days of physiological data collected by IMD 16 from patient 4, determining to send all of the physiological data associated with patient 4 and stored in the memory of IMD 16 to external device 20 may include determining to send the last 30 days of physiological data collected by IMD 16 to external device 20.

In some examples, rather than sending all of the physiological data associated with patient 4 and stored in the memory of IMD 16 to external device 20, ID 16 may send less than all of the physiological data associated with patient 4 and stored in the memory of IMD 16 to external device 20. For example, instead of retransmitting physiological data that has already been previously received by external device 20, IMD 16 may only send physiological data that external device 20 has not previously received.

Instead, IMD 16 may send the physiological data collected by IMD 16 and store in memory after the most recent successful transmission of physiological data to the external device 20. A successful transmission of physiological data may be when all of the physiological data intended to be sent by IMD 16 to external device 20 was successfully sent by IMD 16 and successfully received by external device 20 without any portions of the physiological data intended to be sent by IMD 16 not being successfully sent by IMD 16 and/or not being successfully received by external device 20.

For example, given IMD 16 having memory that stores the last 30 days of physiological data collected by IMD 16, if the last successful transmission of physiological data stored in memory of IMD 16 to the external device 20 occurred 10 days ago, IMD 16 may determine to send the physiological data collected by IMD for the last 10 days. In another example, if the last successful transmission of physiological data stored in memory of IMD 16 to the external device 20 occurred 20 days ago, IMD 16 may determine to send the physiological data previously collected by IMD 16 for the last 20 days.

IMD 16 may associate a timestamp with each piece of physiological data collected by IMD 16 and stored in memory, where the timestamp associated with a piece of physiological data may indicate the date and time at which the piece of physiological data was collected by IMD 16. IMD 16 may store such timestamps associated with the physiological data in memory. IMD 16 may further, in response to a successful transmission of physiological data to external device 20, associate a bookmark with the successful transmission, where the bookmark may be a timestamp that indicates the date and time at which the successful transmission of physiological data occurred. In this way to determine the portion of physiological data in memory.

IMD 16 may therefore compare the timestamps associated with the physiological data stored in memory with the bookmark associated with the most recent successful transmission of physiological data to the external device 20 to determine, out of the physiological data stored in memory, the physiological data collected by IMD 16 after the most recent successful transmission of physiological data to the external device 20. For example, if each timestamp is a concatenation of the four digit year, two digit month, two digit day, and 24-hour time, such as 202106282322 denoting 23:22 on Jun. 28, 2021, IMD 16 may determine that each piece of physiological data having a timestamp that is greater than the timestamp indicated by the bookmark associated with the most recent successful transmission of physiological data to the external device 20 was collected by IMD 16 after the most recent successful transmission of physiological data to the external device 20. In this way, IMD 16 may be able to determine the physiological data in memory that was collected after the most recent successful transmission of physiological data to the external device 20.

In some examples, IMD 16 may determine whether to send, to external device 20, all of the physiological data associated with patient 4 in memory or to send, to external device, the physiological data in memory that was collected after the most recent successful transmission of physiological data to the external device 20 based on the request received from external device 20. For example, external device 20 may send a request for physiological data to IMD 16 that indicates whether the request is a request for all of the physiological data associated with patient 4 in memory or a request for the physiological data in memory that was collected after the most recent successful transmission of physiological data to the external device 20. IMD 16 may therefore determine, based on the request for physiological data from external device 20, whether to send, to external device 20, all of the physiological data associated with patient 4 in memory or to send, to external device, the physiological data in memory that was collected after the most recent successful transmission of physiological data to the external device 20.

In some examples, external device 20 may determine whether to send a request that is a request for all of the physiological data associated with patient 4 in memory or a request that is a request for the physiological data in memory that was collected after the most recent successful transmission of physiological data to the external device 20 and may, in response to making such a determination, send the request to IMD 16. In some examples, as discussed in more detail below, external device 20 may determine the condition of communication link 26 between external device 20 and IMD 16 and may, based on the condition of communication link 26, determine whether to send a request that is a request for all of the physiological data associated with patient 4 in memory or a request that is a request for the physiological data in memory that was collected after the most recent successful transmission of physiological data to the external device 20.

In some examples, external device 20 may receive, from an external server system, such as computing system 24, an indication of whether to send a request that is a request for all of the physiological data associated with patient 4 in memory or a request that is a request for the physiological data in memory that was collected after the most recent successful transmission of physiological data to the external device 20. In some examples, external device 20 may receive user input indicative of whether to send a request that is a request for all of the physiological data associated with patient 4 in memory or a request that is a request for the physiological data in memory that was collected after the most recent successful transmission of physiological data to the external device 20. In some examples, an application executing at external device 20 may call an API function associated with whether to send a request that is a request for all of the physiological data associated with patient 4 in memory or a request that is a request for the physiological data in memory that was collected after the most recent successful transmission of physiological data to the external device 20.

In some examples, when external device 20 sends a request that is a request for the physiological data in memory that was collected after the most recent successful transmission of physiological data to the external device 20, the request may indicate a bookmark associated with a timestamp of the most recent successful transmission of physiological data to the external device 20. IMD 16 may, in response to receiving the request from external device 20, compare the timestamps associated with the physiological data stored in memory with the bookmark indicated by the request to determine the physiological data in memory that was collected after the most recent successful transmission of physiological data to the external device 20.

In some examples, when external device 20 sends a request that is a request for the physiological data in memory that was collected after the most recent successful transmission of physiological data to the external device 20, the request may not indicate a bookmark associated with a timestamp of the most recent successful transmission of physiological data to the external device 20. Instead, IMD 16 may use the bookmark created by IMD 16 and associated with the most recent successful transmission of physiological data to the external device 20 to determine the physiological data in memory that was collected by IMD 16 after the most recent successful transmission of physiological data to the external device 20.

In some examples, IMD 16 may determine whether to send, to external device 20, all of the physiological data associated with patient 4 in memory or to send, to external device, the physiological data in memory that was collected after the most recent successful transmission of physiological data to the external device 20 based on the condition of communication link 26 between external device 20 and IMD 16. The condition of communication link 26 may be based on one or more of the following factors: the signal strength of the wireless connection between external device 20 and IMD 16, the bandwidth of communication link 26, the latency of packets transmitted via communication link 26, the jitter of packets transmitted via communication link 26, packet loss in communication link 26, and/or whether IMD 16 and external device 20 are located in known areas of poor connectivity/high interference, as well as any other indicators of wireless connection quality between IMD 16 and external device 20.

In some examples, IMD 16 may determine whether the condition of communication link 26 between external device 20 and IMD 16 is acceptable for sending, to external device 20, all of the physiological data associated with patient 4 in memory. For example, if IMD 16 determines that the bandwidth of communication link 26 is above a bandwidth threshold and if the packet loss, jitter, and latency are each below a respective threshold, IMD 16 may determine that the condition of communication link 26 is acceptable for sending, to external device 20, all of the physiological data associated with patient 4 in memory and may determine to send, to external device 20, all of the physiological data associated with patient 4 in memory. Conversely, if IMD 16 determines that the bandwidth of communication link 26 is below a bandwidth threshold and/or determines that the bandwidth of communication link 26 is above a bandwidth threshold and if the packet loss, jitter, and latency are each above a respective threshold, IMD 16 may determine that the condition of communication link 26 is not acceptable for sending, to external device 20, all of the physiological data associated with patient 4 in memory and may determine to send, to external device 20, the physiological data in memory that was collected after the most recent successful transmission of physiological data to the external device 20.

In some examples, when external device 20 sends a request for physiological data to MID 16, the request may indicate the condition of communication link 26 between external device 20 and IMD 16. In some examples, the indication of the condition of communication link 26 may indicate whether the condition of the condition of communication link 26 is acceptable for sending, to external device 20, all of the physiological data associated with patient 4 in memory. In some examples, the indication of the condition of communication link 26 may indicate one or more of: the signal strength of the wireless connection between external device 20 and ID 16, the bandwidth of communication link 26, the latency of packets transmitted via communication link 26, the jitter of packets transmitted via communication link 26, packet loss in communication link 26, and/or whether IMD 16 and external device 20 are located in known areas of poor connectivity/high interference, as well as any other indicators of wireless connection quality between IMD 16 and external device 20. MID 16 may determine, based on the condition of communication link 26 as indicated by the request, whether to send, to external device 20, all of the physiological data associated with patient 4 in memory or to send, to external device, the physiological data in memory that was collected after the most recent successful transmission of physiological data to the external device 20.

External device 20 may, in response to receiving the physiological data associated with patient 4 from IMD 16, perform one or more actions to process the physiological data. In some examples, external device 20 may send the physiological data associated with patient 4 and received from IMD 16 to an external computing system, such as computing system 24. In some example, external device 20 may output, for display at a display device, a GUI that includes one or more visualizations of the physiological data received from IMD 16. In some examples, if external device 20 receives the physiological data in memory that was collected after the most recent successful transmission of physiological data to the external device 20, external device 20 may combine such received physiological data with physiological data previously received from IMD 16 and may output, for display at a display device, a GUI that includes one or more visualization of the combined physiological data.

Although external device 20 is illustrated in FIG. 1 as being a single device, in some examples (not illustrated), multiple external device may perform the functions of external device 20.

Although in one example IMD 16 takes the form of an ICM, in other examples, IMD 16 takes the form of any combination of implantable cardioverter defibrillators (ICDs), pacemakers, cardiac resynchronization therapy devices (CRT-Ds), spinal cord stimulation (SCS) devices, deep brain stimulation (DBS) devices, left ventricular assist devices (LVADs), implantable sensors, orthopedic devices, or drug pumps, as examples. Moreover, techniques of this disclosure may be used to communicate with any one of the aforementioned IMDs. Moreover, techniques described in this disclosure may be applied to send and receive physiological data associated with patient 4 between two or more devices, where none of the two or more devices are implantable devices. Additionally, in some examples, techniques described in this disclosure may be applied to send and receive physiological data associated with patient 4 between two or more devices, where none of the two or more devices are medical devices.

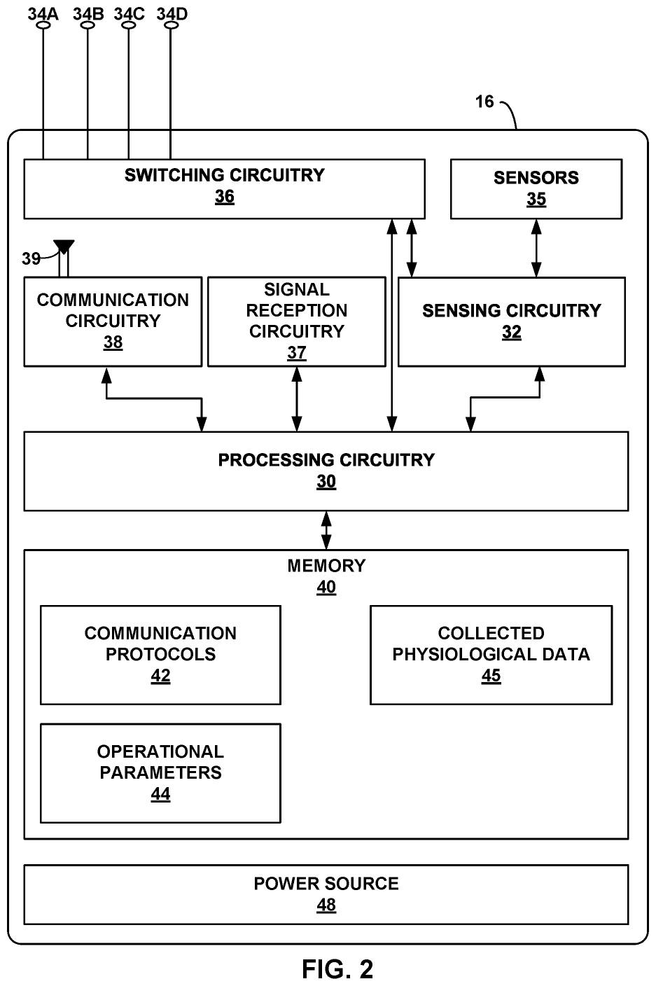

FIG. 2 is a block diagram illustrating an example configuration of components of IMD 16 in accordance with one or more techniques of this disclosure. In the example of FIG. 2, IMD 16 includes processing circuitry 30, sensing circuitry 32, electrodes 34A-34D (collectively, “electrodes 34”), sensors 35, switching circuitry 36, signal reception circuitry 37, communication circuitry 38, antenna 39, memory 40, and power source 48. Memory 40 is configured to store communication protocols 42, operational parameters 44, and/or collected physiological data.

Processing circuitry 30, in one example, may include one or more processors that are configured to implement functionality and/or process instructions for execution within IMD 16. For example, processing circuitry 30 may be capable of processing instructions stored in memory 40. Processing circuitry 30 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 30 may include any suitable structure, whether in hardware, software, firmware, or any combination thereof, to perform the functions ascribed herein to processing circuitry 30.

Sensing circuitry 32 monitors electrical cardiac signals from any combination of electrodes 34A-34D (collectively, “electrodes 34”). In some examples, sensing circuitry 32 may include one or more amplifiers, filters, and analog-to-digital converters. For example, sensing circuitry 32 may include one or more detection channels, each of which may include an amplifier. The detection channels may be used to sense cardiac signals, such as a cardiac EGM. Some detection channels may detect events, such as R-waves, P-waves, and T-waves and provide indications of the occurrences of such events to processing circuitry 30. Additionally, or alternatively, some channels may detect cardiac EGM signals from a particular combination of electrodes 34. One or more other detection channels may provide signals to an analog-to-digital converter, for conversion into a digital signal for processing, analysis, storage, or output by processing circuitry 30.

Each detection channel of sensing circuitry 32 may include a filter configured to pass a custom range of frequency values. For example, sensing circuitry 32 may include one or more narrow band channels, each of which may include a narrow band filtered sense-amplifier. Additionally, or alternatively, sensing circuitry 32 may include one or more wide band channels, each of which include an amplifier with a relatively wider pass band than the narrow band channels. Signals sensed by the narrow band channels and the wide band channels of sensing circuitry 32 may be converted to multi-bit digital signals by an analog-to-digital converter (ADC) provided by, for example, sensing circuitry 32 or processing circuitry 30. In some examples, processing circuitry 30 analyzes the digitized version of signals from sensing circuitry 32. In other examples, processing circuitry 30 stores the digitized versions of the signals in memory 40 (e.g., as collected physiological data 45), outputs the digitized versions of the signals via communication circuitry 38, or any combination thereof.

Processing circuitry 30 may use switching circuitry 36 to select, e.g., via a data/address bus, which of electrodes 34 to use for sensing cardiac signals. Switching circuitry 36 may include a switch array, switch matrix, multiplexer, or any other type of switching device suitable to selectively couple energy to selected electrodes.

In some examples, sensing circuitry 32 is electrically coupled to sensors 35. Sensors 35 may include any combination of accelerometers, temperature sensors, chemical sensors, light sensors, and pressure sensors. Sensors 35 may, for example, sense one or more physiological parameters indicative of a heart condition. Additionally, or alternatively, an accelerometer of sensors 35 may sense data indicative of at least one of patient posture and patient activity.

Signal reception circuitry 37 may include hardware, firmware, software or any combination thereof for receiving signals from another device, such as external device 20. Signal reception circuitry 37 may be powered by power source 48, “listening” for signals from external device 20. In other examples, power source 48 may power signal reception circuitry 37 every 250 milliseconds (ms) for a period of time, where the period of time lasts for greater than 0.1 ms and less than 50 ms. In this way, signal reception circuitry 37 may alternate between an “off” state and an “on” state, where signal reception circuitry 37 is configured to detect signals while signal reception circuitry 37 is being powered by power source 48 during the on state.

Communication circuitry 38 may include any suitable hardware, firmware, software or any combination thereof for communicating with another device, such as external device 20. Under the control of processing circuitry 30, communication circuitry 38 may receive downlink telemetry from, as well as send uplink telemetry to, external device 20 or another device with the aid of an internal or external antenna, e.g., antenna 39. In addition, processing circuitry 30 may communicate with a networked computing device via an external device (e.g., external device 20) and a computer network, such as the Medtronic CareLink® Network developed by Medtronic, plc, of Dublin, Ireland. Communication circuitry 38 may include any combination of a Bluetooth® radio, an electronic oscillator, frequency modulation circuitry, frequency demodulation circuitry, amplifier circuitry, and power switches such as a metal-oxide-semiconductor field-effect transistors (MOSFET), a bipolar junction transistor (BJT), an insulated-gate bipolar transistor (IGBT), a junction field effect transistor (JFET), or another element that uses voltage for its control. Signal reception circuitry 37 may, in some cases, be separate from communication circuitry 38. In other cases, signal reception circuitry 37 may be a component of, or a part of communication circuitry 38.

Memory 40 may be configured to store information within IMD 16 during operation. Memory 40 may include a computer-readable storage medium or computer-readable storage device. In some examples, memory 40 includes one or more of a short-term memory or a long-term memory. Memory 40 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, memory 40 is used to store data indicative of instructions for execution by processing circuitry 30.

In some examples, memory 40 is configured to store one or more communication protocols 42. Each protocol of communication protocols 42 may define a set of rules that govern one or more aspects of data exchange between IMD 16 and other devices (e.g., external device 18 and external device 20. In some examples, communication protocols 42 are stored as lists of computer-readable instructions and communication protocols may be executed by any combination of hardware (e.g., processing circuitry 30) and software. In some examples, communication protocols 42 includes a Bluetooth® protocol such as a BTLE protocol. In some examples, communication protocols 42 exclusively include the Bluetooth® protocol. Alternatively, in other examples, communication protocols 42 may include any combination of Bluetooth® protocols, protocols developed by the manufacturer of IMD 16, and protocols licensed from a third-party developer.

In some examples, memory 40 is configured to store operational parameters 44. Operational parameters 44 may govern aspects of the operation of IMD 16. For example, operational parameters 44 may include combinations of electrodes 34 and sensors 35 for sensing physiological signals of patient 4. Additionally, or alternatively, operational parameters 44 may include a sampling rate for sampling analog signals sensed by electrodes 34 and sensors 35. Operational parameters 44 may be updated based on instructions received from an external device (e.g., external device 20) via communication circuitry 38. In some examples, processing circuitry 30 of IMD 16 updates operational parameters 44 only if instructions to update operational parameters 44 are received over a secure link.

In some examples, memory 40 is configured to store collected physiological data 45. Collected physiological data 45 may include any data sensed, processed, or analyzed by IMD 16, where the data is acquired via any combination of electrodes 34, sensors 35, signal reception circuitry 37 and communication circuitry 38. Such collected physiological data 45 may also be referred to physiological data associated with patient 4 that is collected by IMD 16. In some examples, collected physiological data 45 includes a cardiac EGM recording sensed by sensing circuitry 32 via electrodes 34 and processed by processing circuitry 30. Additionally, or alternatively, collected physiological data 45 may include data acquired by one or more chemical sensors of sensors 35, where the data is indicative of a presence of or a possibility of at least one heart condition (e.g., heart failure). Thus, in general, collected physiological data 45 may represent physiological signals acquired from patient 4 over a period of time and may be referred to as physiological data. In some examples, the period of time lasts for greater than 12 hours and less than 72 hours. In other examples, the period of time lasts for up to one month. Put another way, IMD 16 is configured to continuously monitor physiological signals over the period of time and store at least some of these physiological signals in memory 40 as collected physiological data 45. Memory 40 may store the collected physiological data 45 for a period of time, such as the collected physiological data 45 for the last 30 days, for the last 14 days, for the last week, and the like.

IMD 16 may establish a communication link with another device, such as external device 20. For example, IMD 16 may receive data from external device 20 via communication circuitry 38, and IMD 16 may send data to external device 20 via communication circuitry 38. IMD 16 may send and receive data according to one or more of communication protocols 42. Communication protocols 42 may include one or more protocols and may enable IMD 16 to communicate according to a Bluetooth protocol or a Bluetooth Low Energy protocol, as an example.

Processing circuitry 30 of IMD 16 is configured to send collected physiological data 45 stored in memory 40 to another device, such as external device 20, via communication circuitry 38. Processing circuitry 30 may, in some examples, send collected physiological data 45 to external device 20 without receiving, from external device 20, a request for the collected physiological data 45. In some examples, processing circuitry 30 may receive, from external device 20, a request for collected physiological data 45 and may, in response, send the collected physiological data 45 to external device 20. In some examples, processing circuitry 30 may periodically send collected physiological data 45 to external device 20. In some examples, processing circuitry 30 may send collected physiological data 45 to external device 20 in response to detecting the occurrence of certain conditions in patient 4, such as upon the detecting, via sensors 35 and sensing circuitry 32, the occurrence of an arrhythmia in the heart of patient 4, upon the blood pressure of patient 4 being above or below a specified threshold, upon the heart rate of patient 4 being above or below a specified threshold, and the like.

Processing circuitry 30 may be configured to determine whether to send all of the collected physiological data 45 stored in memory 40 to another device, such as external device 20, or to send a portion (i.e., less than all) of the collected physiological data 45 stored in memory 40. Specifically, processing circuitry 30 may determine whether to send all of the collected physiological data 45 stored in memory 40 to external device 20 or to send the collected physiological data 45 that was collected after the most recent successful transmission of collected physiological data 45 to the external device 20. A successful transmission of collected physiological data 45 to external device 20 may be when all of the collected physiological data 45 intended to be sent by IMD 16 to external device 20 was successfully sent by IMD 16 and successfully received by external device 20 without any portions of the collected physiological data 45 intended to be sent by IMD 16 not being successfully received by external device 20.

Processing circuitry 30 may associate each piece of data in collected physiological data 45 with a timestamp indicative of when (e.g., the time and date) the piece of data was collected by IMD 16 and store the timestamps in memory 40. Processing circuitry 30 may also, in response to a successful transmission of collected physiological data 45 to the external device 20, associate a timestamp indicative of when the successful transmission occurred with the successful transmission. Processing circuitry 30 may therefore determine the most recent timestamp associated with a successful transmission of collected physiological data 45 to the external device 20 as the timestamp associated with the most recent successful transmission of collected physiological data 45 to the external device 20. Processing circuitry 30 may compare the timestamp associated with each piece of data in collected physiological data 45 with the timestamp associated with the most recent successful transmission of collected physiological data 45 to the external device 20 to determine the portion of collected physiological data 45 that was collected after the most recent successful transmission of collected physiological data 45 to the external device 20.

In some examples, IMD 16 may receive, from external device 20, a request for collected physiological data 45 that indicates whether the request is a request for all of collected physiological data 45 stored in memory 40 or whether the request is a request for the portion of collected physiological data 45 that was collected after the most recent successful transmission of collected physiological data 45 to the external device 20. Processing circuitry 30 may therefore determine, based on the request, whether to send all of the collected physiological data 45 stored in memory 40 to external device 20, or to send the portion of collected physiological data 45 that was collected after the most recent successful transmission of collected physiological data 45 to the external device 20. Processing circuitry 30 may therefore send, to external device 20, either all of the collected physiological data 45 stored in memory 40 or the portion of collected physiological data 45 that was collected after the most recent successful transmission of collected physiological data 45 to the external device 20 based on such determination.

In some examples, Processing circuitry 30 may determine whether to send all of the collected physiological data 45 stored in memory 40 to external device 20, or to send, to external device 20, the portion of collected physiological data 45 that was collected after the most recent successful transmission of collected physiological data 45 to the external device 20 based at least in part on the connection quality between IMD 16 and external device 20. In some examples, IMD 16 may receive, from external device 20, a request for collected physiological data 45 that indicates the connection quality between IMP 16 and external device 20, and processing circuitry 30 may determine, based on the connection quality between ID 16 and external device 20 indicated by the request, whether to send, to external device 20, all of the collected physiological data 45 stored in memory 40 to external device 20, or to send, to external device 20, the portion of collected physiological data 45 that was collected after the most recent successful transmission of collected physiological data 45 to the external device 20.

In some examples, if the request received from external device 20 indicates that the connection quality is acceptable for IMP 16 to send, to external device 20, all of the collected physiological data 45 stored in memory 40, processing circuitry 30 may correspondingly send, to external device 20, all of the collected physiological data 45 stored in memory 40. Conversely, if the request received from external device 20 indicates that the connection quality is not acceptable for IMD 16 to send, to external device 20, all of the collected physiological data 45 stored in memory 40, processing circuitry 30 may send, to external device 20, the portion of collected physiological data 45 that was collected after the most recent successful transmission of collected physiological data 45 to the external device 20.

In some examples, processing circuitry 30 may determine, based on one or more factors associated with the connection quality between external device 20 and IMD 16, the connection quality between external device 20 and IMD 16 and may determine, based on the connection quality. whether to send, to external device 20, all of the collected physiological data 45 stored in memory 40 to external device 20, or to send, to external device 20, the portion of collected physiological data 45 that was collected after the most recent successful transmission of collected physiological data 45 to the external device 20. The one or more factors associated with the connection quality may include one or more of the following factors: the signal strength of the connection between external device 20 and IMD 16, the bandwidth of the connection, the latency of packets transmitted via the connection, the jitter of packets transmitted via the connection, packet loss in the connection and/or whether IMD 16 and external device 20 are located in known areas of poor connectivity/high interference, as well as any other indicators of wireless connection quality between IMD 16 and external device 20.

In some examples, processing circuitry 30 may use communication circuitry 38 to periodically determine the values of these one or more factors associated with the connection quality between external device 20 and IMD 16 to determine the connection quality. In some examples, processing circuitry 30 may receive, from external device 20, a request that indicates the one or more factors associated with the connection quality between external device 20 and IMD 16. Processing circuitry 30 may determine, based on the one or more factors associated with the connection quality between external device 20 and IMD 16, whether the connection quality is acceptable for IMD 16 to send, to external device 20, all of the collected physiological data 45 stored in memory 40. For example, if the bandwidth of the connection between external device 20 and IMD 16 is below a bandwidth threshold and if the latency of packets transmitted via the connection, the jitter of packets transmitted via the connection, packet loss in the connection are each above a threshold, processing circuitry 30 may determine that the connection quality is not acceptable for IMD 16 to send, to external device 20, all of the collected physiological data 45 stored in memory 40. Conversely, if the bandwidth of the connection between external device 20 and IMD 16 is above a bandwidth threshold and if the latency of packets transmitted via the connection, the jitter of packets transmitted via the connection, packet loss in the connection are each below a threshold, processing circuitry 30 may determine that the connection quality is acceptable for IMD 16 to send, to external device 20, all of the collected physiological data 45 stored in memory 40.

Power source 48 is configured to deliver operating power to the components of IMD 16. Power source 48 may include a battery and a power generation circuit to produce the operating power. In some examples, the battery is rechargeable to allow extended operation. In some examples, recharging is accomplished through proximal inductive interaction between an external charger and an inductive charging coil within external device 18. Power source 48 may include any one or more of a plurality of different battery types, such as nickel cadmium batteries and lithium ion batteries.

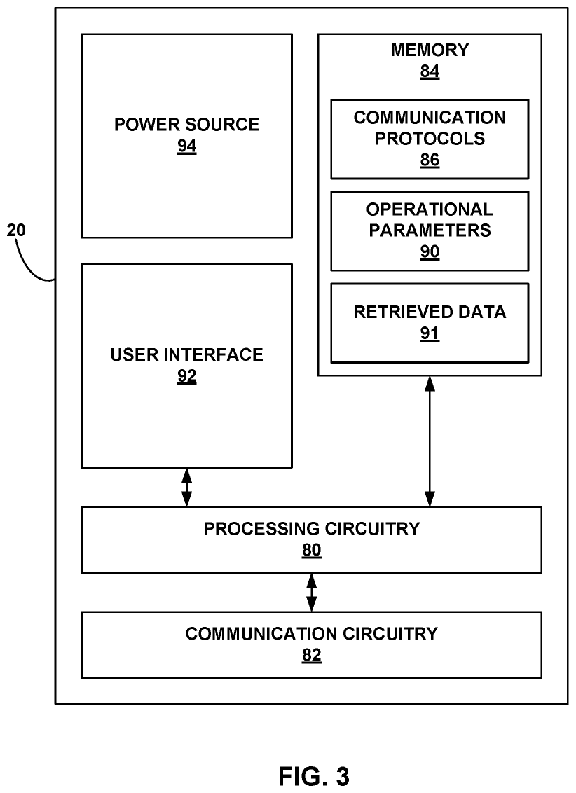

FIG. 3 is a block diagram illustrating an example configuration of components of external device 20 in accordance with one or more techniques of this disclosure. In the example of FIG. 3, external device 20 includes processing circuitry 80, communication circuitry 82, memory 84, user interface 92, and power source 94. Memory 84 is configured to store communication protocols 86, operational parameters 90, and retrieved data 91.

Processing circuitry 80, in one example, may include one or more processors that are configured to implement functionality and/or process instructions for execution within external device 20. For example, processing circuitry 80 may be capable of processing instructions stored in memory 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD. Under the control of processing circuitry 80, communication circuitry 82 may receive downlink telemetry from, as well as send uplink telemetry to, IMD 16 or another device. In some examples, communication circuitry 82 includes a first set of communication circuitry configured for transmitting and receiving signals according to a communication protocol developed by the manufacturer of IMD 16 or a third-party developer. In some such examples, communication circuitry 82 further includes a second set of communication circuitry which defines a Bluetooth radio configured for transmitting and receiving signals according to Bluetooth communication protocols, including Bluetooth Low Energy protocols. However, communication circuitry 82 does not necessarily include separate sets of circuitry corresponding to different communication protocols. In some examples, communication circuitry 82 includes a single set of circuitry configured for transmitting and receiving signals according to a plurality of communication protocols.

In some examples, communication circuitry 82 includes any combination of a Bluetooth radio, an electronic oscillator, frequency modulation circuitry, frequency demodulation circuitry, amplifier circuitry, and power switches such as a MOSFET, a BJT, an IGBT, a JFET, or another element that uses voltage for its control.

Memory 84 may be configured to store information within external device 20 during operation. Memory 84 may include a computer-readable storage medium or computer-readable storage device. In some examples, memory 84 includes one or more of a short-term memory or a long-term memory. Memory 84 may include, for example, RAM, DRAM, SRAM, magnetic discs, optical discs, flash memories, or forms of EPROM or EEPROM. In some examples, memory 84 is used to store data indicative of instructions for execution by processing circuitry 80. Memory 84 may be used by software or applications running on external device 20 to temporarily store information during program execution.

External device 20 may exchange information with other devices via communication circuitry 82 according to one or more communication protocols 86. Communication protocols 86, stored in memory 84, may include sets of computer-readable instructions that determine how data is transmitted and processed. Communication protocols 86 may include one or more communication protocols that are additionally included in each of communication protocols 42 and communication protocols 68. In other words, IMD 16, and external device 20 may be configured to exchange information according to at least one common communication protocol. In some examples, the one or more common communication protocols include at least one Bluetooth communication protocol. Additionally, or alternatively, communication protocols 86 may include a set of communication protocols that are not available to IMD 16. In some examples, external device 20 is a consumer electronics device, such as a smartphone, a tablet, or a laptop computer. In some such examples, external device 20 may not be configured with communication protocols developed by the manufacturer of IMD 16.

Data exchanged between external device 20 and IMD 16 may also include any of operational parameters 90 stored in memory 84. External device 20 may transmit data including computer readable instructions which, when implemented by IMD 16, may control IMD 16 to change one or more operational parameters according to operational parameters 90 and/or export collected data. For example, processing circuitry 80 may transmit an instruction to IMD 16 which requests IMD 16 to export collected data (e.g., a portion of collected physiological data 45) to external device 20. In turn, external device 20 may receive the collected data from IMD 16 and store the collected data in memory 84 (e.g., as retrieved data 91). Additionally, or alternatively, processing circuitry 80 may export instructions to IMD 16 requesting IMD 16 to update electrode combinations for stimulation or sensing according to operational parameters 90.

External device 20 may be configured to send, to IMD 16 and via communication circuitry 82, requests for physiological data associated with patient 4 and collected by IMID 16 (e.g., collected physiological data 45). In some examples, external device 20 may send, to IMD 16, a request for physiological data associated with patient 4 that indicates whether the request is a request for all of the physiological data associated with patient 4 and stored in memory of IMD 16 or whether the request is a request for the portion of the physiological data collected by IMD 16 and stored in memory of IMD 16 after the most recent successful transmission of collected physiological data from IMD 16 to the external device 20.

In some examples, processing circuitry 80 may be configured to determine to send, to IMD 16, a request for collected physiological data 45. In some examples, processing circuitry 80 may periodically send, to IMD 16, requests for collected physiological data 45, such as each hour, each day, and the like. In some examples, processing circuitry 80 may receive, from an external server system, such as computing system 24, and via communication circuitry 82, a request for collected physiological data 45 and may, in response, send a request for collected physiological data 45 to IMD 16. In some examples, user interface 92 may receive user input indicative of a request for collected physiological data 45 and may, in response, send a request for collected physiological data 45 to TN/ID 16. In some examples, processing circuitry 80 may determine that an application executing at processing circuitry 80 has called an API function to send a request for collected physiological data 45 to ID 16 and may, in response, send a request for collected physiological data 45 to IMD 16.

Processing circuitry 80 may be configured to determine whether to send, to IMD 16, a request for all of collected physiological data 45 stored in memory of IMD 16 or to send, to IMD 16, a request for the portion of collected physiological data 45 stored in memory of IMD 16 collected by IMP 16 after the most recent successful transmission of collected physiological data from IMD 16 to the external device 20. In some examples, processing circuitry 80 may determine whether to send a request for all of collected physiological data 45 stored in memory of IMD 16 or to send a request for the portion of collected physiological data 45 stored in memory of IMD 16 collected by IMD 16 after the most recent successful transmission of collected physiological data from ID 16 to the external device 20 based on the connection quality between IMD 16 and external device 20.

Processing circuitry 80 may determine whether the connection quality of the connection between IMD 16 and external device 20 is acceptable for ID 16 to send all of the collected physiological data 45 to external device 20. If processing circuitry 80 determines that the connection quality of the connection between IMD 16 and external device 20 is acceptable for IMD 16 to send all of the collected physiological data 45 to external device 20, processing circuitry 80 may send, to IMP 16, a request for all of collected physiological data 45 stored in memory of IMP 16. If processing circuitry 80 determines that the connection quality of the connection between IMD 16 and external device 20 is not acceptable for ID 16 to send all of the collected physiological data 45 to external device 20, processing circuitry 80 may send, to IMD 16, a request for the portion of collected physiological data 45 stored in memory of IMD 16 collected by IMD 16 after the most recent successful transmission of collected physiological data from ID 16 to the external device 20.

In some examples, processing circuitry 80 may determine, based on one or more factors associated with the connection quality between external device 20 and ID 16, the connection quality between external device 20 and ID 16 and may determine, based on the connection quality, whether to send a request for all of collected physiological data 45 stored in memory of IMD 16 or to send a request for the portion of collected physiological data 45 stored in memory of IMP 16 collected by IMP 16 after the most recent successful transmission of collected physiological data from IMD 16 to the external device 20. The one or more factors associated with the connection quality may include one or more of the following factors: the signal strength of the connection between external device 20 and IMD 16, the bandwidth of the connection, the latency of packets transmitted via the connection, the jitter of packets transmitted via the connection, packet loss in the connection and/or whether IMD 16 and external device 20 are located in known areas of poor connectivity/high interference, as well as any other indicators of wireless connection quality between ID 16 and external device 20.

In some examples, processing circuitry 80 may use communication circuitry 38 to periodically determine the values of these one or more factors associated with the connection quality between external device 20 and IMD 16 to determine the connection quality. For example, if the bandwidth of the connection between external device 20 and IMD 16 is below a bandwidth threshold and if the latency of packets transmitted via the connection, the jitter of packets transmitted via the connection, packet loss in the connection are each above a threshold, processing circuitry 30 may determine that the connection quality is not acceptable to send a request for all of collected physiological data 45 stored in memory of IMD 16. Conversely, if the bandwidth of the connection between external device 20 and ID 16 is above a bandwidth threshold and if the latency of packets transmitted via the connection, the jitter of packets transmitted via the connection, packet loss in the connection are each below a threshold, processing circuitry 30 may determine that the connection quality is acceptable to send a request for all of collected physiological data 45 stored in memory of IMD 16.

In some examples, processing circuitry 80 may not send, to ID 16, a request that indicates whether the request is a request for all of collected physiological data 45 stored in memory of IMD 16 or whether the request is a request for the portion of collected physiological data 45 stored in memory of IMD 16 collected by IMD 16 after the most recent successful transmission of collected physiological data from ID 16 to the external device 20. Instead, processing circuitry 80 may send, to ID 16, a request for collected physiological data 45 that indicates the connection quality of the connection between external device 20 and IMD 16. In some examples, the connection quality of the connection between external device 20 and ID 16 indicated by the request may be an indication of whether the connection quality is acceptable to send a request for all of collected physiological data 45 stored in memory of IMD 16. In some examples, the connection quality of the connection between external device 20 and IMD 16 may indicate one or more of the following factors associated with the connection between external device 20 and IMD 16: the signal strength of the connection between external device 20 and IMD 16, the bandwidth of the connection, the latency of packets transmitted via the connection, the jitter of packets transmitted via the connection, packet loss in the connection and/or whether IMD 16 and external device 20 are located in known areas of poor connectivity/high interference, as well as any other indicators of wireless connection quality between IMD 16 and external device 20.

Correspondingly, external device 20 may receive, from IMD 16 and via communication circuitry 82, physiological data associated with patient 4 that may be either all of the physiological data associated with patient 4 and stored in memory of IMD 16 or the portion of the physiological data collected by IMD 16 and stored in memory of IMD 16 after the most recent successful transmission of collected physiological data from IMD 16 to the external device 20. External device 20 may store the physiological data received from IMD 16 in memory 84 as retrieved data 91. Processing circuitry 80 may also perform one or more actions to process the retrieved data 91. In some examples, processing circuitry 80 may send retrieved data 91 to an external computing system, such as computing system 24. In some example, processing circuitry 80 may output, for display at a display device, a GUI that includes one or more visualizations of the retrieved data 91. In some examples, if external device 20 receives the physiological data that was collected after the most recent successful transmission of physiological data to the external device 20, processing circuitry 80 may combine such received physiological data with physiological data previously received from IMD 16 and stored as retrieved data 91 and may output, for display at a display device in user interface 92, a GUI that includes one or more visualization of the combined physiological data.

A user, such as a clinician or patient 4, may interact with external device 20 through user interface 92. User interface 92 includes a display (not shown), such as an LCD or LED display or other type of screen, with which processing circuitry 80 may present information related to IMD 16 (e.g., EGM signals obtained from at least one electrode or at least one electrode combination). In addition, user interface 92 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 20 and provide input. In other examples, user interface 92 also includes audio circuitry for providing audible notifications, instructions or other sounds to patient 4, receiving voice commands from patient 4, or both. Memory 84 may include instructions for operating user interface 92 and for managing power source 94.

Power source 94 is configured to deliver operating power to the components of external device 20. Power source 94 may include a battery and a power generation circuit to produce the operating power. In some examples, the battery is rechargeable to allow extended operation. Recharging may be accomplished by electrically coupling power source 94 to a cradle or plug that is connected to an alternating current (AC) outlet. In addition, recharging may be accomplished through proximal inductive interaction between an external charger and an inductive charging coil within external device 20. In other examples, traditional batteries (e.g., nickel cadmium or lithium ion batteries) may be used. In addition, external device 20 may be directly coupled to an alternating current outlet to operate.

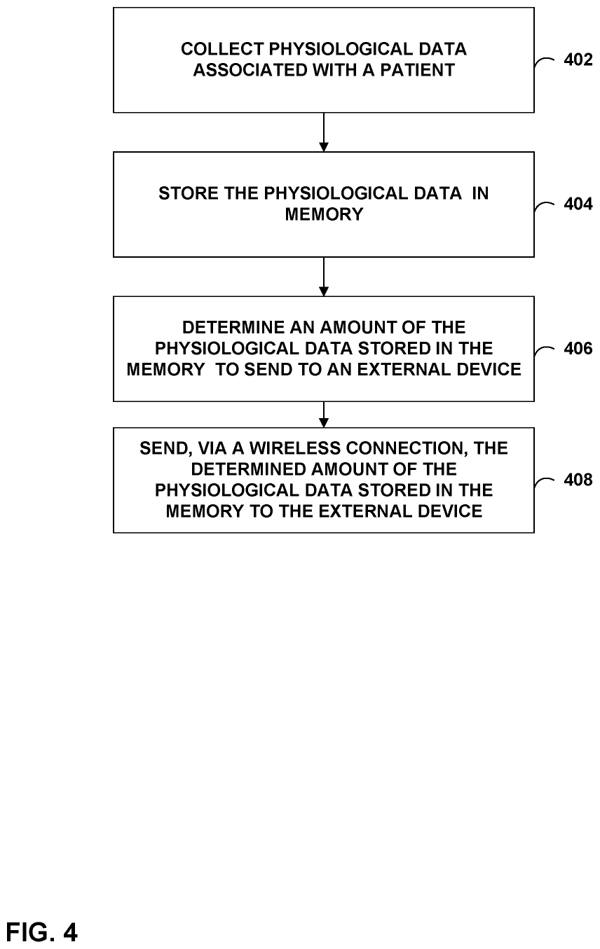

FIG. 4 is a flow diagram illustrating an example operation in accordance with one or more techniques of this disclosure. The example operation is described with respect to IMD 16 and external device 20 of FIGS. 1-3, and components thereof.

As shown in FIG. 4, processing circuitry 30 of medical device 16 may collect physiological data associated with a patient 4 (402). Processing circuitry 30 may store the physiological data in memory 40 (404). Processing circuitry 30 may determine an amount of the physiological data 45 stored in the memory 40 to send to an external device 20 (406). Processing circuitry 30 may send, via a wireless connection 26, the determined amount of the physiological data stored in the memory 40 to the external device 20 (408).

In some examples, processing circuitry 30 may determine whether to send all of the physiological data 45 stored in the memory 40 to the external device 20 or to send a portion of the physiological data 45 collected after a most recent successful transmission of previously collected physiological data 45 to the external device 20.

In some examples, processing circuitry 30 may determine whether to send all of the physiological data 45 stored in the memory 40 to the external device 20 or to send a portion of the physiological data 45 collected after a most recent successful transmission of previously collected physiological data 45 to the external device 20 based at least in part on a connection quality of the wireless connection 26 between the medical device 16 and the external device 20.

In some examples, processing circuitry 30 may determine the connection quality of the wireless connection 26 between the medical device 16 and the external device 20 based at least in part on one or more factors associated with the connection quality.

In some examples, processing circuitry 30 may, in response to determining that the connection quality of the wireless connection 26 between the medical device 16 and the external device 20 is acceptable for sending all of the physiological data 45 stored in the memory to the external device 20, determine to send all of the physiological data 45 stored in the memory 40 to the external device 20.

In some examples, processing circuitry 30 may, in response to determining that the connection quality of the wireless connection 26 between the medical device 16 and the external device 20 is not acceptable for sending all of the physiological data 45 stored in the memory 40 to the external device 20, determine to send the portion of the physiological data 45 collected after the most recent successful transmission of the previously collected physiological data 45 to the external device 20.

In some examples, processing circuitry 30 may receive, from the external device 20, a request for the physiological data 45 stored in the memory 40. Moreover, processing circuitry 30 may determine, based on the request, whether to send all of the physiological data 45 stored in the memory 40 to the external device 20 or to send the portion of the physiological data 45 collected after the most recent successful transmission of previously collected physiological data 45 to the external device 20.

In some examples, processing circuitry 30 may, in response to determining to send all of the physiological data stored in the memory 45 to the external device 20, send, via the wireless connection 26, all of the physiological data 45 stored in the memory 40 to the external device 20.

In some examples, processing circuitry 30 may, in response to determining to send the portion of the physiological data 45 collected after the most recent successful transmission of the previously collected physiological data 45 to the external device 20, send, via the wireless connection 26, the portion of the physiological data 45 collected after the most recent successful transmission of the previously collected physiological data 45 to the external device 20.

In some examples, processing circuitry 30 may compare timestamps associated with the physiological data 45 stored in the memory 40 with a timestamp associated with the most recent successful transmission of the previously collected physiological data 45 to the external device 20 to determine the portion of the physiological data 45 collected after the most recent successful transmission of the previously collected physiological data 45 to the external device 20.

Aspects of this disclosure includes the following examples.

Example 1: A method includes collecting, by processing circuitry of a medical device, physiological data associated with a patient; storing, by the processing circuitry, the physiological data in memory; determining, by the processing circuitry, an amount of the physiological data stored in the memory to send to an external device; and sending, by the processing circuitry via a wireless connection, the determined amount of the physiological data stored in memory to the external device.

Example 2: The method of example 1, wherein determining the amount of the physiological data stored in memory to send to the external device further comprises: determining, by the processing circuitry, whether to send all of the physiological data stored in the memory to the external device or to send a portion of the physiological data collected after a most recent successful transmission of previously collected physiological data to the external device.

Example 3: The method of example 2, wherein determining whether to send all of the physiological data stored in the memory to the external device or to send the portion of the physiological data collected after the most recent successful transmission of previously collected physiological data to the external device further comprises: determining, by the processing circuitry, whether to send all of the physiological data stored in the memory to the external device or to send a portion of the physiological data collected after a most recent successful transmission of previously collected physiological data to the external device based at least in part on a connection quality of the wireless connection between the medical device and the external device.

Example 4: The method of example 3, further includes determining, by the processing circuitry, the connection quality of the wireless connection between the medical device and the external device based at least in part on one or more factors associated with the connection quality.

Example 5: The method of any of examples 3 and 4, wherein determining whether to send all of the physiological data stored in the memory to the external device or to send a portion of the physiological data collected after a most recent successful transmission of previously collected physiological data to the external device based at least in part on the connection quality of the wireless connection between the medical device and the external device further comprises: determining, by the processing circuitry, that the connection quality of the wireless connection between the medical device and the external device is acceptable for sending all of the physiological data stored in the memory to the external device; and in response to determining that the connection quality of the wireless connection between the medical device and the external device is acceptable for sending all of the physiological data stored in the memory to the external device, determining, by the processing circuitry, to send all of the physiological data stored in the memory to the external device.

Example 6: The method of any of examples 3 and 4, wherein determining whether to send all of the physiological data stored in the memory to the external device or to send a portion of the physiological data collected after a most recent successful transmission of previously collected physiological data to the external device based at least in part on the connection quality of the wireless connection between the medical device and the external device further comprises: determining, by the processing circuitry, that the connection quality of the wireless connection between the medical device and the external device is not acceptable for sending all of the physiological data stored in the memory to the external device; and in response to determining that the connection quality of the wireless connection between the medical device and the external device is not acceptable for sending all of the physiological data stored in the memory to the external device, determining, by the processing circuitry, to send the portion of the physiological data collected after the most recent successful transmission of the previously collected physiological data to the external device.

Example 7: The method of any of examples 2 through 6, wherein determining whether to send all of the physiological data stored in the memory to the external device or to send the portion of the physiological data collected after the most recent successful transmission of previously collected physiological data to the external device further comprises: receiving, by the processing circuitry and from the external device, a request for the physiological data stored in the memory; and determining, by the processing circuitry and based on the request, whether to send all of the physiological data stored in the memory to the external device or to send the portion of the physiological data collected after the most recent successful transmission of previously collected physiological data to the external device.

Example 8: The method of any of examples 2 through 7, wherein sending the determined amount of the physiological data stored in memory to the external device further comprises: in response to determining to send all of the physiological data stored in the memory to the external device, sending, by the processing circuitry via the wireless connection, all of the physiological data stored in the memory to the external device.

Example 9: The method of any of examples 2 through 8, wherein sending the determined amount of the physiological data stored in memory to the external device further comprises: in response to determining to send the portion of the physiological data collected after the most recent successful transmission of the previously collected physiological data to the external device, sending, by the processing circuitry via the wireless connection, the portion of the physiological data collected after the most recent successful transmission of the previously collected physiological data to the external device.

Example 10: The method of any of examples 2-9, further includes comparing, by the processing circuitry, timestamps associated with the physiological data stored in the memory with a timestamp associated with the most recent successful transmission of the previously collected physiological data to the external device to determine the portion of the physiological data collected after the most recent successful transmission of the previously collected physiological data to the external device.

Example 11: The method of any of examples 1-10, wherein the wireless connection comprises a Bluetooth Low Energy connection.

Example 12: A medical device configured for wireless communication, wherein the medical device comprises: sensing circuitry configured to collect physiological data associated with a patient; memory configured to store the physiological data; communication circuitry configured for wireless communication; and processing circuitry electrically coupled to the communication circuitry, wherein the processing circuitry is configured to: determine an amount of the physiological data stored in the memory to send to an external device; and send, using the communication circuitry and via a wireless connection, the determined amount of the physiological data stored in memory to the external device.

Example 13: The medical device of example 12, wherein to determine the amount of the physiological data stored in memory to send to the external device, the processing circuitry is further configured to: determine whether to send all of the physiological data stored in the memory to the external device or to send a portion of the physiological data collected after a most recent successful transmission of previously collected physiological data to the external device.

Example 14: The medical device of example 13, wherein to determine whether to send all of the physiological data stored in the memory to the external device or to send the portion of the physiological data collected after the most recent successful transmission of previously collected physiological data to the external device, the processing circuitry is further configured to: determining, by the processing circuitry, whether to send all of the physiological data stored in the memory to the external device or to send a portion of the physiological data collected after a most recent successful transmission of previously collected physiological data to the external device based at least in part on a connection quality of the wireless connection between the medical device and the external device.

Example 15: The medical device of example 14, wherein the processing circuitry is further configured to: determine the connection quality of the wireless connection between the medical device and the external device based at least in part on one or more factors associated with the connection quality.

Example 16: The medical device of any of examples 14 and 15, wherein to determine whether to send all of the physiological data stored in the memory to the external device or to send a portion of the physiological data collected after a most recent successful transmission of previously collected physiological data to the external device based at least in part on the connection quality of the wireless connection between the medical device and the external device, the processing circuitry is further configured to: determine that the connection quality of the wireless connection between the medical device and the external device is acceptable for sending all of the physiological data stored in the memory to the external device; and in response to determining that the connection quality of the wireless connection between the medical device and the external device is acceptable for sending all of the physiological data stored in the memory to the external device, determine to send all of the physiological data stored in the memory to the external device.

Example 17: The medical device of any of examples 14 and 15, wherein to determine whether to send all of the physiological data stored in the memory to the external device or to send a portion of the physiological data collected after a most recent successful transmission of previously collected physiological data to the external device based at least in part on the connection quality of the wireless connection between the medical device and the external device, the processing circuitry is further configured to: determine that the connection quality of the wireless connection between the medical device and the external device is not acceptable for sending all of the physiological data stored in the memory to the external device; and in response to determining that the connection quality of the wireless connection between the medical device and the external device is not acceptable for sending all of the physiological data stored in the memory to the external device, determine to send the portion of the physiological data collected after the most recent successful transmission of the previously collected physiological data to the external device.

Example 18: The medical device of any of examples 13 through 17, wherein to determine whether to send all of the physiological data stored in the memory to the external device or to send the portion of the physiological data collected after the most recent successful transmission of previously collected physiological data to the external device, the processing circuitry is further configured to: receive, from the external device, a request for the physiological data stored in the memory; and determine, based on the request, whether to send all of the physiological data stored in the memory to the external device or to send the portion of the physiological data collected after the most recent successful transmission of previously collected physiological data to the external device.

Example 19: The medical device of any of examples 13 through 18, wherein to send the determined amount of the physiological data stored in memory to the external device, the processing circuitry is further configured to: in response to determining to send all of the physiological data stored in the memory to the external device, send, using the communication circuitry and via the wireless connection, all of the physiological data stored in the memory to the external device.

Example 20: The medical device of any of examples 13 through 19, wherein to send the determined amount of the physiological data stored in memory to the external device, the processing circuitry is further configured to: in response to determining to send the portion of the physiological data collected after the most recent successful transmission of the previously collected physiological data to the external device, send, using the communication circuitry and via the wireless connection, the portion of the physiological data collected after the most recent successful transmission of the previously collected physiological data to the external device.

Example 21: The medical device of any of examples 13-20, the processing circuitry is further configured to: compare, timestamps associated with the physiological data stored in the memory with a timestamp associated with the most recent successful transmission of the previously collected physiological data to the external device to determine the portion of the physiological data collected after the most recent successful transmission of the previously collected physiological data to the external device.

Example 22: The medical device of any of examples 12-21, wherein the wireless connection comprises a Bluetooth Low Energy connection.

Example 23: An apparatus comprising means for performing any of the methods of examples 1-11.

Example 24: A non-transitory computer-readable storage medium comprising program instructions that, when executed by processing circuitry of a medical device, cause the processing circuitry to perform the methods of any of examples 1-11.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an ID, an external programmer, a combination of an ID and external programmer, an integrated circuit (IC) or a set of ICs, and/or discrete electrical circuitry, residing in an IMD and/or external programmer.

What is claimed is:

1. A method comprising:
collecting, by processing circuitry of a medical device, physiological data associated with a patient;
storing, by the processing circuitry, the physiological data in memory;
determining, by the processing circuitry, an amount of the physiological data stored in the memory to send to an external device; and
sending, by the processing circuitry via a wireless connection, the determined amount of the physiological data stored in memory to the external device.

2. The method of claim 1, wherein determining the amount of the physiological data stored in memory to send to the external device further comprises:
determining, by the processing circuitry, whether to send all of the physiological data stored in the memory to the external device or to send a portion of the physiological data collected after a most recent successful transmission of previously collected physiological data to the external device.

3. The method of claim 2, wherein determining whether to send all of the physiological data stored in the memory to the external device or to send the portion of the physiological data collected after the most recent successful transmission of previously collected physiological data to the external device further comprises:
determining, by the processing circuitry, whether to send all of the physiological data stored in the memory to the external device or to send a portion of the physiological data collected after a most recent successful transmission of previously collected physiological data to the external device based at least in part on a connection quality of the wireless connection between the medical device and the external device.

4. The method of claim 3, wherein determining whether to send all of the physiological data stored in the memory to the external device or to send a portion of the physiological data collected after a most recent successful transmission of previously collected physiological data to the external device based at least in part on the connection quality of the wireless connection between the medical device and the external device further comprises:
determining, by the processing circuitry, that the connection quality of the wireless connection between the medical device and the external device is acceptable for sending all of the physiological data stored in the memory to the external device; and
in response to determining that the connection quality of the wireless connection between the medical device and the external device is acceptable for sending all of the physiological data stored in the memory to the external device, determining, by the processing circuitry, to send all of the physiological data stored in the memory to the external device.

5. The method of claim 3, wherein determining whether to send all of the physiological data stored in the memory to the external device or to send a portion of the physiological data collected after a most recent successful transmission of previously collected physiological data to the external device based at least in part on the connection quality of the wireless connection between the medical device and the external device further comprises:
determining, by the processing circuitry, that the connection quality of the wireless connection between the medical device and the external device is not acceptable for sending all of the physiological data stored in the memory to the external device; and
in response to determining that the connection quality of the wireless connection between the medical device and the external device is not acceptable for sending all of the physiological data stored in the memory to the external device, determining, by the processing circuitry, to send the portion of the physiological data collected after the most recent successful transmission of the previously collected physiological data to the external device.

6. The method of claim 2, wherein determining whether to send all of the physiological data stored in the memory to the external device or to send the portion of the physiological data collected after the most recent successful transmission of previously collected physiological data to the external device further comprises:
receiving, by the processing circuitry and from the external device, a request for the physiological data stored in the memory; and
determining, by the processing circuitry and based on the request, whether to send all of the physiological data stored in the memory to the external device or to send the portion of the physiological data collected after the most recent successful transmission of previously collected physiological data to the external device.

7. The method of claim 2, wherein sending the determined amount of the physiological data stored in memory to the external device further comprises:
in response to determining to send all of the physiological data stored in the memory to the external device, sending, by the processing circuitry via the wireless connection, all of the physiological data stored in the memory to the external device.

8. The method of claim 2, wherein sending the determined amount of the physiological data stored in memory to the external device further comprises:
in response to determining to send the portion of the physiological data collected after the most recent successful transmission of the previously collected physiological data to the external device, sending, by the processing circuitry via the wireless connection, the portion of the physiological data collected after the most recent successful transmission of the previously collected physiological data to the external device.

9. The method of claim 2, further comprising:
comparing, by the processing circuitry, timestamps associated with the physiological data stored in the memory with a timestamp associated with the most recent successful transmission of the previously collected physiological data to the external device to determine the portion of the physiological data collected after the most recent successful transmission of the previously collected physiological data to the external device.

10. The method of claim 1, wherein the wireless connection comprises a Bluetooth Low Energy connection.

11. A medical device configured for wireless communication, wherein the medical device comprises:
sensing circuitry configured to collect physiological data associated with a patient;
memory configured to store the physiological data;
communication circuitry configured for wireless communication; and
processing circuitry electrically coupled to the communication circuitry, wherein the processing circuitry is configured to:
determine an amount of the physiological data stored in the memory to send to an external device; and
send, using the communication circuitry and via a wireless connection, the determined amount of the physiological data stored in memory to the external device.

12. The medical device of claim 11, wherein to determine the amount of the physiological data stored in memory to send to the external device, the processing circuitry is further configured to:
determine whether to send all of the physiological data stored in the memory to the external device or to send a portion of the physiological data collected after a most recent successful transmission of previously collected physiological data to the external device.

13. The medical device of claim 12, wherein to determine whether to send all of the physiological data stored in the memory to the external device or to send the portion of the physiological data collected after the most recent successful transmission of previously collected physiological data to the external device, the processing circuitry is further configured to:
determining, by the processing circuitry, whether to send all of the physiological data stored in the memory to the external device or to send a portion of the physiological data collected after a most recent successful transmission of previously collected physiological data to the external device based at least in part on a connection quality of the wireless connection between the medical device and the external device.

14. The medical device of claim 13, wherein to determine whether to send all of the physiological data stored in the memory to the external device or to send a portion of the physiological data collected after a most recent successful transmission of previously collected physiological data to the external device based at least in part on the connection quality of the wireless connection between the medical device and the external device, the processing circuitry is further configured to:
determine that the connection quality of the wireless connection between the medical device and the external device is acceptable for sending all of the physiological data stored in the memory to the external device; and
in response to determining that the connection quality of the wireless connection between the medical device and the external device is acceptable for sending all of the physiological data stored in the memory to the external device, determine to send all of the physiological data stored in the memory to the external device.

15. The medical device of claim 13, wherein to determine whether to send all of the physiological data stored in the memory to the external device or to send a portion of the physiological data collected after a most recent successful transmission of previously collected physiological data to the external device based at least in part on the connection quality of the wireless connection between the medical device and the external device, the processing circuitry is further configured to:
determine that the connection quality of the wireless connection between the medical device and the external device is not acceptable for sending all of the physiological data stored in the memory to the external device; and
in response to determining that the connection quality of the wireless connection between the medical device and the external device is not acceptable for sending all of the physiological data stored in the memory to the external device, determine to send the portion of the physiological data collected after the most recent successful transmission of the previously collected physiological data to the external device.

16. The medical device of claim 12, wherein to determine whether to send all of the physiological data stored in the memory to the external device or to send the portion of the physiological data collected after the most recent successful transmission of previously collected physiological data to the external device, the processing circuitry is further configured to:
receive, from the external device, a request for the physiological data stored in the memory; and
determine, based on the request, whether to send all of the physiological data stored in the memory to the external device or to send the portion of the physiological data collected after the most recent successful transmission of previously collected physiological data to the external device.

17. The medical device of claim 12, wherein to send the determined amount of the physiological data stored in memory to the external device, the processing circuitry is further configured to:
in response to determining to send all of the physiological data stored in the memory to the external device, send, using the communication circuitry and via the wireless connection, all of the physiological data stored in the memory to the external device.

18. The medical device of claim 12, wherein to send the determined amount of the physiological data stored in memory to the external device, the processing circuitry is further configured to:
in response to determining to send the portion of the physiological data collected after the most recent successful transmission of the previously collected physiological data to the external device, send, using the communication circuitry and via the wireless connection, the portion of the physiological data collected after the most recent successful transmission of the previously collected physiological data to the external device.

19. The medical device of claim 12, the processing circuitry is further configured to:
compare, timestamps associated with the physiological data stored in the memory with a timestamp associated with the most recent successful transmission of the previously collected physiological data to the external device to determine the portion of the physiological data collected after the most recent successful transmission of the previously collected physiological data to the external device.

20. A non-transitory computer-readable storage medium comprising program instructions that, when executed by processing circuitry of a medical device, cause the processing circuitry to:
collect physiological data associated with a patient;
store the physiological data in memory;
determine an amount of the physiological data stored in the memory to send to an external device; and
send, via a wireless connection, the determined amount of the physiological data stored in memory to the external device.
